(19) United States
(12) Patent Application Publication
(10) Pub. No.: US 2020/0143943 A1
(43) Pub. Date: May 7, 2020

(54) DECISION SUPPORT SYSTEM FOR NOTIFYING OF VARIANCES

(71) Applicant: International Business Machines Corporation, Armonk, NY (US)

(72) Inventors: Justin C. Nelson, Rochester, MN (US); Sarah N. Ulmer, Rochester, MN (US); Alexandra L. Dorenkamp, Rochester, MN (US); Quinton G. Kramer, Rochester, MN (US); Jim C. Chen, Rochester, MN (US)

(21) Appl. No.: 16/179,003

(22) Filed: Nov. 2, 2018

(51) Int. Cl. G16H 50/20; G06N 5/04

(52) U.S. Cl. CPC G16H 50/20; G06N 5/045

(57) ABSTRACT

A decision support engine allows defining multiple events and corresponding actions. An event could be, for example, a decision that is at variance with what the professional usually suggests, or at variance with standard practice of other professionals. Suitable action can be taken when an event occurs. Examples of suitable actions include, for example, to notify the professional, to notify the customer or patient, to notify an administrator, and to log all data related to the decision that caused the event. The decision support engine receives biometric input from one or more biometric sensors worn by the professional, and can thus log the physical state of the professional at the time the event occurs. For each event, the professional's biometric data is logged and can be analyzed to determine statistical correlations between the events corresponding to the professional and the professional's physical state.

### BACKGROUND

### 1. Technical Field

This disclosure generally relates to decision support systems, and more specifically relates to a system that provides notifications of variances in decisions.

### 2. Background Art

Experts and professionals are subject to the same life conditions as the average person: stress, fatigue, life-disrupting events, distractions, etc. These life conditions can lead to decisions that are not consistent with their normal practice, or in some cases, even inconsistent with what a consensus of other experts would provide.

### BRIEF SUMMARY

A decision support engine allows defining multiple events and corresponding actions for those events. An event could be, for example, a decision that is at variance with what the professional usually suggests, or at variance with standard practice of other professionals. Suitable action can be taken when an event occurs. Examples of suitable actions include, for example, to notify the professional, to notify the customer or patient, to notify an administrator, and to log all data related to the decision that caused the event. The decision support engine receives biometric input from one or more biometric sensors worn by the professional, and can thus log the physical state of the professional at the time the event occurs. Both conforming and non-conforming decisions can be logged to a profile maintained for the professional, along with the corresponding biometric data. For each event, the professional's biometric data is logged and can be analyzed to determine statistical correlations between the events corresponding to the professional and the professional's physical state.

The foregoing and other features and advantages will be apparent from the following more particular description, as illustrated in the accompanying drawings.

### BRIEF DESCRIPTION OF THE SEVERAL VIEWS OF THE DRAWING(S)

The disclosure will be described in conjunction with the appended drawings, where like designations denote like elements, and:

FIG. 1 is a block diagram of a computer system that includes a decision support engine;

FIG. 2 is a block diagram of a specific configuration for a system that includes a medical decision support engine that is one specific embodiment for the decision support engine shown in FIG. 1;

FIG. 3 is a block diagram showing possible biometric data for a professional that could be collected;

FIG. 4 is a block diagram showing possible input sensors;

FIG. 5 is a block diagram showing suitable examples of sensor data;

FIG. 6 is a block diagram showing possible data that could be included in a professional profile for a professional;

FIG. 7 is a block diagram showing three exemplary severity levels for the purpose of illustration;

FIG. 8 is a block diagram showing possible actions that can be taken in response to an event;

FIG. 9 is a flow diagram of a method for defining events and corresponding severity levels and/or corresponding actions;

FIG. 10 is a table showing three examples of event types;

FIG. 11 is a table showing correlation between sample events, corresponding severity levels, and corresponding actions;

FIG. 12 is a table showing correlation between events and severity levels;

FIG. 13 is a table showing correlation between severity levels and corresponding actions;

FIG. 14 is a flow diagram of a method for automatically performing actions corresponding to an event when the event occurs;

FIG. 15 is a flow diagram of a method for the medical decision support engine to log information relating to an event;

FIG. 16 is a flow diagram of a method for generating and logging a physical health score for a diagnosis or treatment that caused an event;

FIG. 17 is a flow diagram of a method for generating a competency score for a medical professional based on data logged in the medical professional's profile; and

FIG. 18 is a flow diagram for analyzing and correlating a medical professional's physical state to events.

### DETAILED DESCRIPTION

The disclosure and claims herein are directed to a decision support engine that allows defining multiple events and corresponding actions for those events. An event could be, for example, a decision that is at variance with what the professional usually suggests, or at variance with standard practice of other professionals. Suitable action can be taken when an event occurs. Examples of suitable actions include, for example, to notify the professional, to notify the customer or patient, to notify an administrator, and to log all data related to the decision that caused the event. The decision support engine receives biometric input from one or more biometric sensors worn by the professional, and can thus log the physical state of the professional at the time the event occurs. Both conforming and non-conforming decisions can be logged to a profile maintained for the professional, along with the corresponding biometric data. For each event, the professional's biometric data is logged and can be analyzed to determine statistical correlations between the events corresponding to the professional and the professional's physical state.

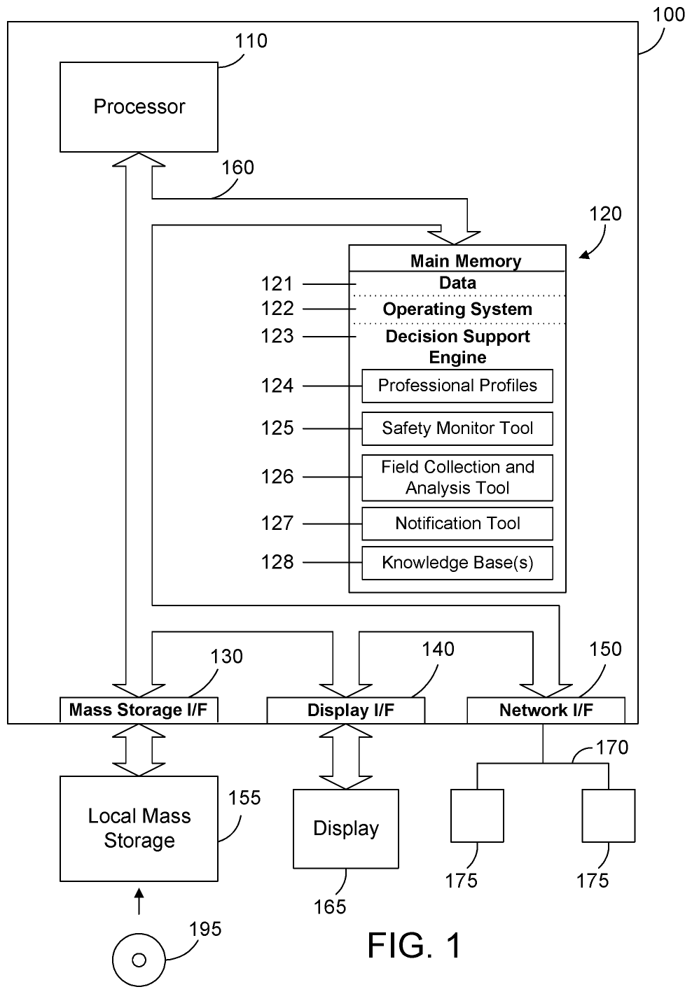

Referring to FIG. 1, a computer system 100 is one suitable implementation of a computer system that includes a decision support engine as described in more detail below. Computer system 100 is an IBM POWER8 computer system. However, those skilled in the art will appreciate that the disclosure herein applies equally to any computer system, regardless of whether the computer system is a complicated multi-user computing apparatus, a single user workstation, a laptop computer system, a tablet computer, a phone, or an embedded control system. As shown in FIG. 1, computer system 100 comprises one or more processors 110, a main memory 120, a mass storage interface 130, a display interface 140, and a network interface 150. These system components are interconnected through the use of a system bus 160. Mass storage interface 130 is used to connect mass storage devices, such as local mass storage device 155, to computer system 100. One specific type of local mass storage device 155 is a readable and writable CD-RW drive, which may store data to and read data from a CD-RW 195. Another suitable type of local mass storage device 155 is a card reader that receives a removable memory card, such as an SD card, and performs reads and writes to the removable memory. Yet another suitable type of local mass storage device 155 is universal serial bus (USB) that reads a storage device such as a flash drive.

Main memory 120 preferably contains data 121, an operating system 122, and a decision support engine 123. Data 121 represents any data that serves as input to or output from any program in computer system 100. Operating system 122 is a multitasking operating system, such as AIX or LINUX. Decision support engine 123 defines multiple events and corresponding actions. The decision support engine 123 receives data from many sources, including biometric data corresponding to professionals, and determines when a decision varies enough to cause one of the events. When an event occurs, the event and corresponding physical state of the professional is logged.

The decision support engine 123 includes professional profiles 124, a safety monitor tool 125, a field collection and analysis tool 126, a notification tool 127, and one or more knowledge bases 128. The professional profiles 124 may include any suitable information relating to a professional. Professional profiles 124 may include, for example, events and the professional's corresponding physical state when the event occurs. The safety monitor tool 125 receives biometric data for one or more professionals. The biometric data for a professional can be logged when an event occurs. The field collection and analysis tool 126 includes one or more input sensors that log sensor data corresponding to a professional, a patient, or the environment. The notification tool 127 provides one or more prescribed notifications when an event occurs. Knowledge base(s) 128 can include any suitable database of information that could be accessed by the decision support engine. Suitable examples of knowledge bases 128 could include, for example, a knowledge base that includes work and rest regulations for professionals; a medical cases knowledge base that includes symptoms and usual treatments for those symptoms; and a medical professional case handling knowledge base that includes logged diagnoses and treatments, and may optionally include one or more logged conditions corresponding to the logged decisions.

Computer system 100 utilizes well known virtual addressing mechanisms that allow the programs of computer system 100 to behave as if they only have access to a large, contiguous address space instead of access to multiple, smaller storage entities such as main memory 120 and local mass storage device 155. Therefore, while data 121, operating system 122, and decision support engine 123 are shown to reside in main memory 120, those skilled in the art will recognize that these items are not necessarily all completely contained in main memory 120 at the same time. It should also be noted that the term “memory” is used herein generically to refer to the entire virtual memory of computer system 100, and may include the virtual memory of other computer systems coupled to computer system 100.

Processor 110 may be constructed from one or more microprocessors and/or integrated circuits. Processor 110 executes program instructions stored in main memory 120. Main memory 120 stores programs and data that processor 110 may access. When computer system 100 starts up, processor 110 initially executes the program instructions that make up operating system 122. Processor 110 also executes the decision support engine 123.

Although computer system 100 is shown to contain only a single processor and a single system bus, those skilled in the art will appreciate that a decision support engine as described herein may be practiced using a computer system that has multiple processors and/or multiple buses. In addition, the interfaces that are used preferably each include separate, fully programmed microprocessors that are used to off-load compute-intensive processing from processor 110. However, those skilled in the art will appreciate that these functions may be performed using I/O adapters as well.

Display interface 140 is used to directly connect one or more displays 165 to computer system 100. These displays 165, which may be non-intelligent (i.e., dumb) terminals or fully programmable workstations, are used to provide system administrators and users the ability to communicate with computer system 100. Note, however, that while display interface 140 is provided to support communication with one or more displays 165, computer system 100 does not necessarily require a display 165, because all needed interaction with users and other processes may occur via network interface 150.

Network interface 150 is used to connect computer system 100 to other computer systems or workstations 175 via network 170. Computer systems 175 represent computer systems that are connected to the computer system 100 via the network interface 150 in a computer cluster. Network interface 150 broadly represents any suitable way to interconnect electronic devices, regardless of whether the network 170 comprises present-day analog and/or digital techniques or via some networking mechanism of the future. Network interface 150 preferably includes a combination of hardware and software that allows communicating on the network 170. Software in the network interface 150 preferably includes a communication manager that manages communication with other computer systems 175 via network 170 using a suitable network protocol. Many different network protocols can be used to implement a network. These protocols are specialized computer programs that allow computers to communicate across a network. TCP/IP (Transmission Control Protocol/Internet Protocol) is an example of a suitable network protocol that may be used by the communication manager within the network interface 150. In one suitable implementation, the network interface 150 is a physical Ethernet adapter.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

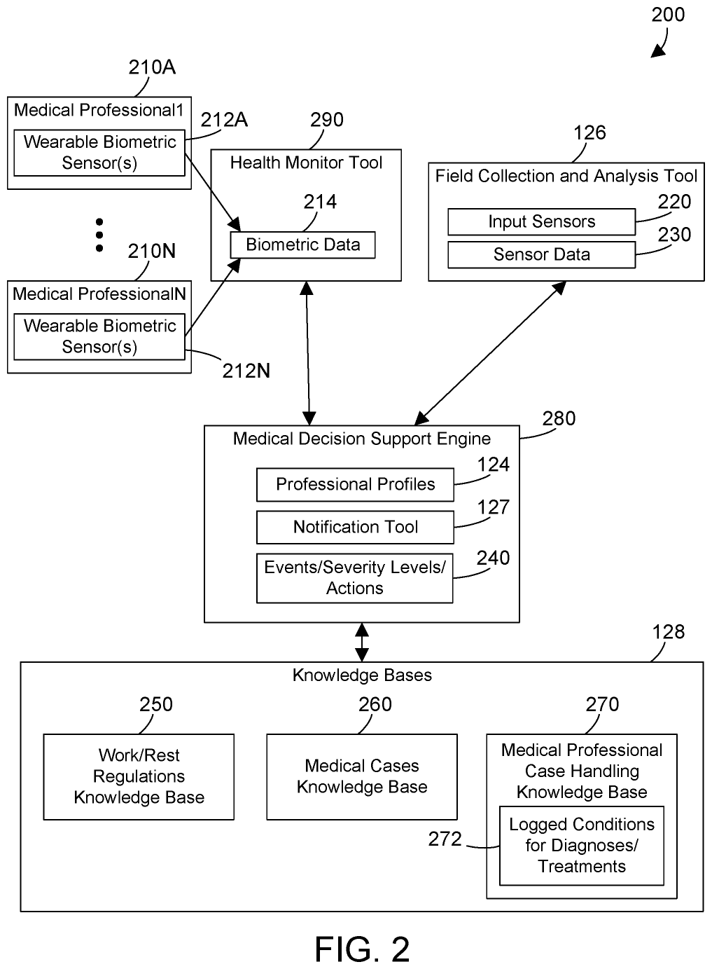

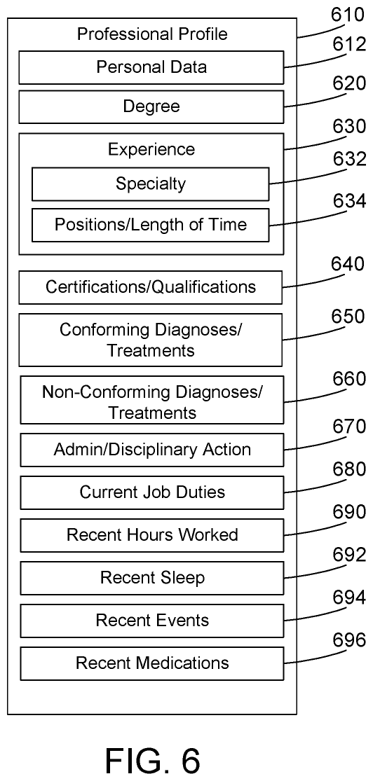

FIG. 2 shows one sample system 200 within the scope of the disclosure and claims herein. Note that different portions of system 200 in FIG. 2 could reside on different computer systems, including distributed computer systems in a cloud computing environment. System 200 includes a medical decision support engine 280 that is one suitable embodiment of the decision support engine 123 shown in FIG. 1, with various components shown in different locations. The medical decision support engine 280 preferably includes the professional profiles 124 and notification tool 127 shown in FIG. 1. The professional profiles 124 may include any suitable information. Some examples of suitable information that could be included in a professional profile are shown in FIG. 6. The notification tool 127 provides notification of an event to one or more persons, as described in more detail below. The medical decision support engine 280 additionally includes defined events, which can have corresponding severity levels and/or actions 240. The events and corresponding severity levels and/or actions 240 are preferably defined by a human user or administrator, but could be generated in other suitable ways. In one specific embodiment, the events 240 include one or more corresponding actions, and do not include a severity level. In another specific embodiment, each event 240 includes both a severity level as well as the one or more corresponding actions.

The medical decision support engine 123 preferably queries databases referred to herein as knowledge bases 128, which can include a work/rest regulations knowledge base 250, a medical cases knowledge base 260, and a medical profession case handling knowledge base 270. The work/rest regulations knowledge base 250 could be, for example, a database for a hospital that specified hospital policies regarding number of hours a medical professional is allowed to work within a given time period, required hours of sleep between shifts, number of consecutive shifts allowed, etc. The medical cases knowledge base 260 preferably includes a database of symptoms and acceptable diagnoses and treatments for those systems. The medical cases knowledge base 260 most preferably represents medical cases and corresponding symptoms, diagnoses and treatment from a large number of medical professionals, such as thousands or tens of thousands. The medical professional case handling knowledge base 270 includes medical cases, with their corresponding symptoms, diagnoses and treatments, for medical professionals that have a professional profile 124 in the medical decision support engine 280. The medical professional case handling knowledge based 270 preferably includes logged conditions for diagnoses and treatments 272. The logged conditions for diagnoses/treatments can include biometric data 214 received from the health monitor tool, as well as sensor data 230 received from the field collection and analysis tool 126. In this manner a medical professional's diagnoses and treatments are tied to the physical condition of the medical professional when the diagnoses and treatments were made, thereby allowing statistical analysis to be performed to determine whether there are correlations between variances in the diagnoses and treatments of a medical professional and the physical state of the medical professional, as indicated by the corresponding biometric data and sensor data.

The safety monitor tool 125 receives biometric data 214 from one or more wearable biometric sensors worn by professionals. One specific embodiment of safety monitor tool 125 in FIG. 1 is the health monitor tool 290 shown in FIG. 2, which receives biometric data from multiple medical professionals, including medical professional 210A who wears one or more corresponding biometric sensors 212A, . . . , medical professional 210N, who wears one or more corresponding biometric sensors 212N. The biometric data 214 preferably includes biometric data from all medical professionals that have a professional profile 124 in the medical decision support engine 280, where each medical professional's biometric data is correlated to that medical professional. Because the medical decision support engine 280 correlates biometric data 214 and sensor data 230 with diagnoses and treatments, these conditions may be logged as logged conditions for diagnoses/treatments 272 in the medical professional case handling knowledge base 128.

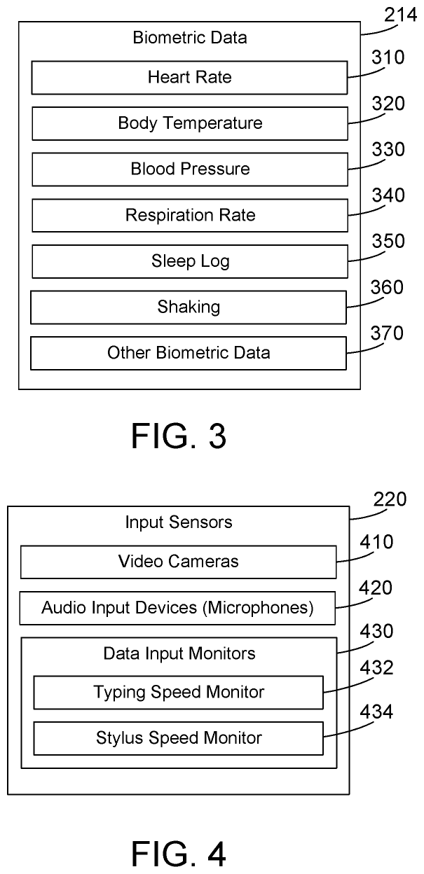

Examples of suitable biometric data 214 that could be collected for medical professionals are shown in FIG. 3, and include: heart rate 310; body temperature 320; blood pressure 330; respiration rate 340, sleep log 350; shaking 360; and other biometric data 370. The measuring of heart rate 310, body temperature 320, blood pressure 330, respiration rate 340, and shaking 360 could be done, for example, by a wearable sensor the medical professional wears on a wrist like a watch, or by one or more sensors placed on the skin at suitable locations. A wearable biometric monitor can monitor when a person is asleep or awake, and can thus generate sleep logs 350 for a person. The sleep log 350 can be used as biometric data that is attached to a particular diagnosis or treatment. Other biometric data 370 can include any suitable biometric data for a medical professional that can be measured using any known or future device. For example, other biometric devices could collect blood sugar and glycerin levels as well.

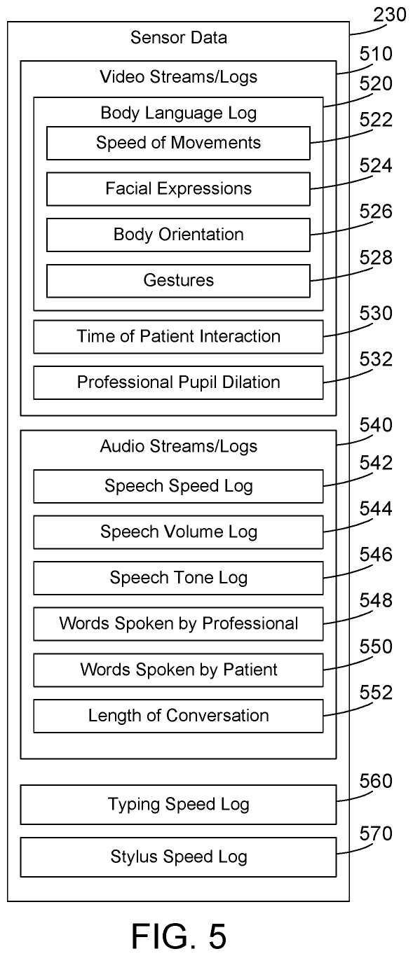

Referring again to FIG. 2, the field collection and analysis tool 126 includes input sensors 220 that generate sensor data 230 that is read by the medical decision support engine 280. The input sensors 220 could be distributed over many different physical areas and over many different systems. Examples of suitable input sensors 220 are shown in FIG. 4, and include: video cameras 410; audio input devices (or microphones) 420; and data input monitors 430. Data input monitors 430 could include, for example, typing speed monitor 432 and stylus speed monitor 434. If a medical professional is in an unusual physical state, the medical professional could type more slowly or more quickly than usual, or the medical professional could use a stylus more slowly or more quickly than usual. The data input monitors 430 can thus help detect when something is unusual about the medical professional. The input sensors 220 generate suitable sensor data 230 that can be read by the medical decision support engine 280 to determine physical state of a medical professional and/or patient. Suitable data generated by input sensors 220 can include, for example, video streams/logs, audio streams/logs, typing speed and stylus speed, as shown in more detail in FIG. 5.

Examples of suitable sensor data 230 that can be generated by the input sensors 220 are shown in FIG. 5, and include: video streams/logs 510; audio streams/logs 540; typing speed log 560; and stylus speed log 570. Video streams/logs 510 can be analyzed to determine speed of movements 522, facial expressions 524, body orientation 526, and gestures 528, of both medical professionals and patients. Of course, other data not specifically identified in FIG. 5 could also be analyzed and determined from the video streams/logs 510. The time of patient interaction 530 can also be determined from the video streams/logs 510. The pupil dilation of the professional 532 could also be determined when the video stream/log provides sufficient view of the eyes of the medical professional. The body language log 520 could include other data not specifically shown in FIG. 5. One example of such other data is the rate of blinking for the medical professional.

Audio streams/logs 540 can be analyzed to determine speech speed log 542, speech volume log 544, speech tone log 546, words spoken by the professional 548, words spoken by the patient 550; and the length of conversation 552. Of course, other data not specifically identified in FIG. 5 could also be analyzed and determined from the audio streams/logs 540. The typing speed log 560 includes a log of all data entered by any or all of the medical professionals via a keyboard or keypad, which allows determining typing speed for each interaction of a medical professional using a keyboard or keypad. The stylus speed log 570 includes a log of all data entered by any or all of the medical professionals via a stylus, which allows determining stylus speed for each interaction of a medical professional using a stylus. Other analysis could also be performed, such as accuracy of typing and handwriting analysis from input from a stylus.

The decision support engine 123 shown in FIG. 1 and the medical decision support engine 280 in FIG. 2 preferably include professional profiles 124. Referring to FIG. 6, one specific professional profile 610 is shown to illustrate data that could be included in a professional profile within the scope of the disclosure and claims herein. The professional profile 610 in FIG. 6 includes: personal data 612; degree 620; experience 630, which can include specialty 632 and positions with corresponding length of time 634; certifications/qualifications 640; conforming diagnoses/treatments 650; non-conforming diagnoses/treatments 660; administrative or disciplinary action 670; current job duties 680; recent hours worked 690; recent sleep 692; recent events 694; and recent medications 696. Personal data 610 could include any suitable personal information, such as height, weight, address, phone number, etc. Degree 620 indicates the degree the medical professional received in school. Experience 630 can include the medical professional's specialty 632 along with positions the medical professional has held with their corresponding length of time 634. Certifications/qualifications 640 specify any certifications or qualifications for the medical professional, such as being a board-certified thoracic surgeon, or having qualified as a trauma surgeon.

Conforming diagnoses/treatments 650 are those diagnoses and treatments by the medical professional that were not at variance with accepted diagnoses and treatments, and which resulted in good results for the patient. Bad diagnoses/treatments 660 are those diagnoses by the medical professional that were at variance with accepted diagnoses and treatments, and which resulted in bad results for the patient. Note a variance in a diagnosis or treatment does not mean the diagnosis or treatment is a bad diagnosis or treatment, because the medical professional may be intentionally varying from accepted standards, believing the circumstances of the patient justify a different approach. The professional profile 610 thus could include a third category of diagnoses and treatments not shown in FIG. 6, diagnoses and treatments that are at variance from accepted practice, but for this the outcome for the patient was good. In the alternative, diagnoses and treatments in this category could be included in the conforming diagnoses/treatments 650.

Admin/Disciplinary action 670 can indicate whether the medical professional has had any administrative or disciplinary action by any suitable entity, including the medical professional's current employer, or any suitable association or organization. Current job duties 680 specify the job duties of the medical professional. Thus, the certifications/qualifications for a medical professional might specify the medical professional is a trauma nurse, while the current job duties 680 might indicate duties other than emergency room trauma. Recent hours worked 690 could be from a database that shows the shifts the medical professional has recently worked. Recent sleep 692 could be from a sleep log, such as 350 in FIG. 3, retrieved from a wearable sensor worn by the medical professional. Recent events 694 could include any suitable life event that could affect a person's work, such as having a new baby, which typically causes disruption in sleep for the parents, recent return from vacation, which may make it hard for a medical professional to get back in the groove, a recent death in the family, etc. Recent medications 696 could include medications taken by the medical professional. For example, if the medical professional had a recent event 694 of a car accident that caused a painful injury, for which the medical professional is taking pain medication, the pain medication could be listed in recent medications 696.

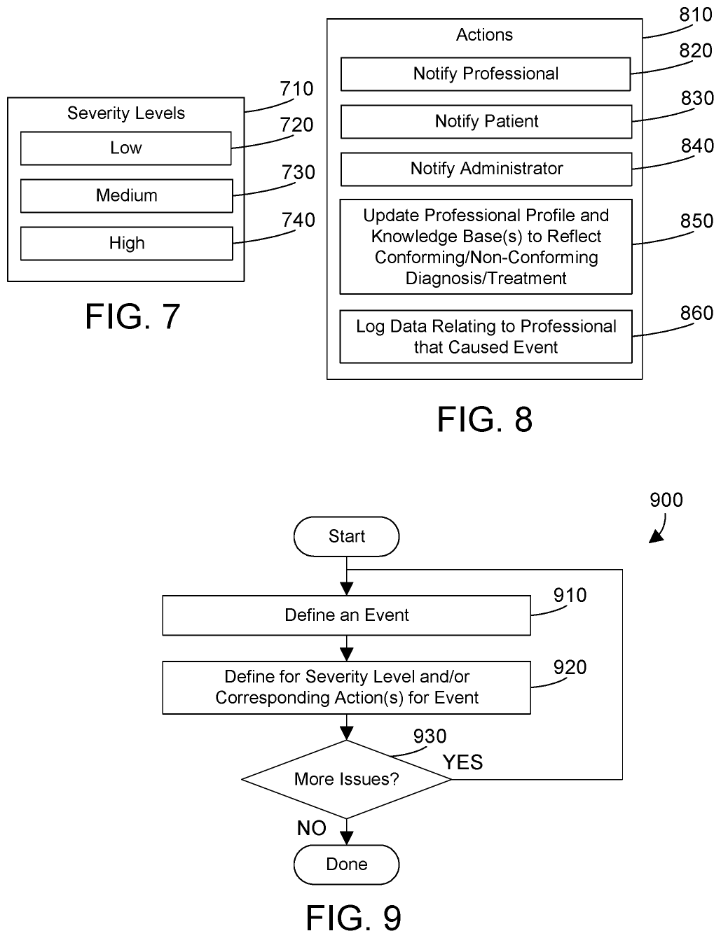

Referring to FIG. 7, one suitable example for severity levels 710 show severity levels of low 720, medium 730 and high 740. Different actions can be specified according to the severity level, as discussed below with reference to FIGS. 11 and 13.

An event can specify to take any suitable action when the event occurs. Examples of suitable actions 810 are shown in FIG. 8, and include: notify the medical professional 820. notify the patient 830; notify an administrator 840; update the professional profile and one or more knowledge bases to reflect a conforming or non-conforming diagnosis and/or treatment 850, and log data relating to the professional that caused the event 860. Of course, any suitable action besides those shown in FIG. 8 could be included in an event within the scope of the disclosure and claims herein, including notifying the medical professional with recommended steps for the professional to take.

A method 900 in FIG. 9 is preferably performed by the decision support engine 123 shown in FIG. 1 and/or the medical decision support engine 280 shown in FIG. 2. An event is defined (step 910). A corresponding severity level and/or action(s) for the event are defined (step 920). When there are more issues for which events need to be defined (step 930=YES), method 900 loops back to step 910 and continues until there are no more issues for which events need to be defined (step 930=NO). Method 900 is then done.

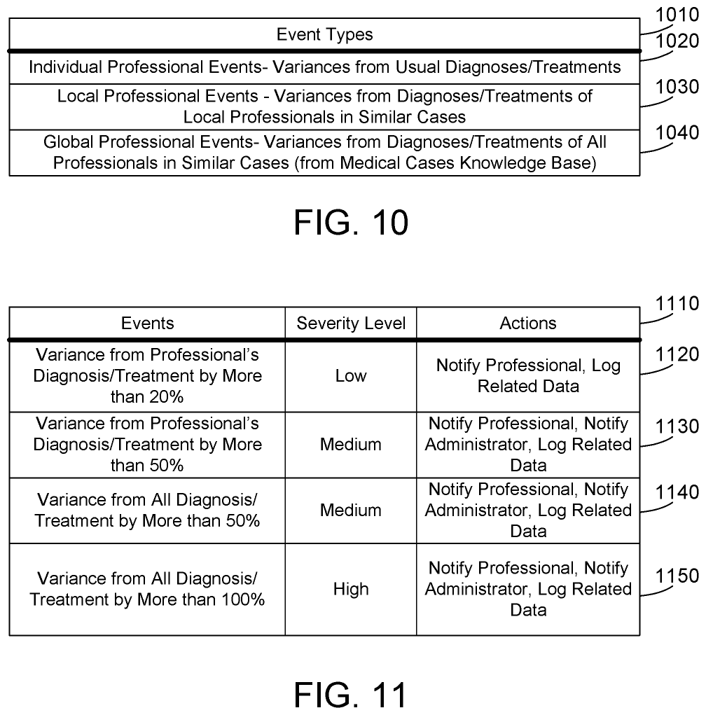

Various types of events could be defined within the scope of the disclosure and claims herein. Referring to FIG. 10, three examples of event types are shown in table 1010, and include: individual professional events, which are variances from a medical professional's usual diagnoses and/or treatments 1020; local professional events, which are variances from diagnoses and/or treatments of local professionals in similar cases 1030; and global professional events, which are variances from diagnoses/treatments for all professionals in similar cases 1040, which can be retrieved, for example, from the medical cases knowledge base 260 shown in FIG. 2. Note the term “local” in 1030 is used to mean medical professionals within a particular organization, such as a hospital or group of hospitals owned by the same company. The term “global” in 1040 does not necessarily mean global in the geographical sense, but is used to include medical professionals that are outside of a particular organization, such as a hospital or group of hospitals owned by the same company. In the most preferred implementation, global professional events are based on diagnoses and treatments that are well-accepted by most medical professionals in the industry.

FIG. 11 shows a table 1110 with four sample events that each has a corresponding severity level and one or more actions. The first event in table 1110 specifies a variance from a medical professional's diagnosis or treatment by more than twenty percent, which has a severity level of “low” and corresponding actions of “notify professional” and “log related data”, as shown in entry 1120. The second event in table 1110 specifies a variance from a medical professional's diagnosis or treatment by more than fifty percent, which has a severity level of “medium” and corresponding actions of “notify professional”, “notify an administrator” and “log related data”, as shown in entry 1130. The third event in table 1110 specifies a variance from diagnosis/treatment of all medical professionals by more than fifty percent, which has a severity level of “medium” and corresponding actions of “notify professional”, “notify an administrator” and “log related data”, as shown in entry 1140. The fourth event in table 1110 specifies a variance from diagnosis/treatment of all medical professionals by more than one hundred percent, which has a severity level of “high” and corresponding actions of “notify professional”, “notify an administrator” and “log related data”, as shown in entry 1150.

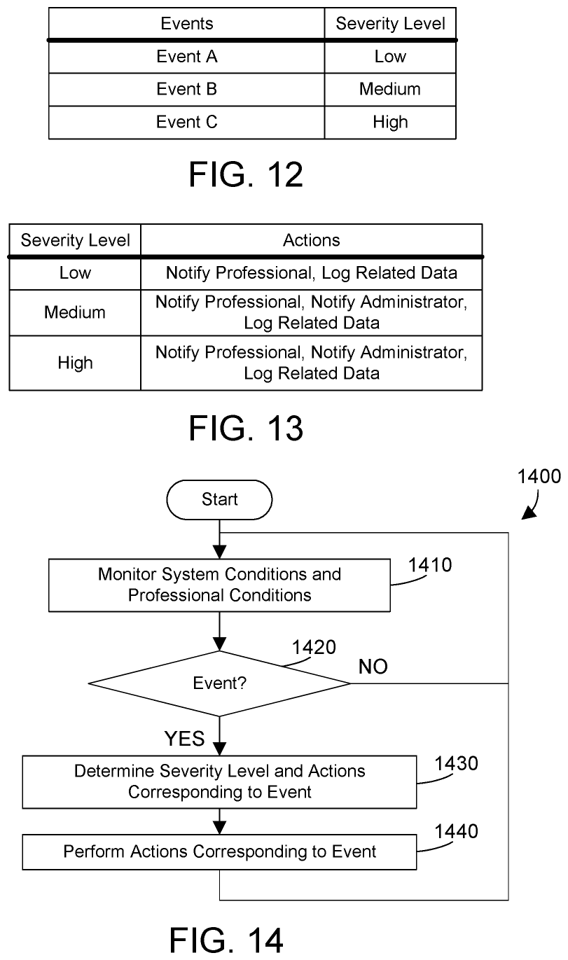

Instead of defining a severity level and corresponding actions for each event, as shown in FIG. 11, an alternative is to define a severity level for each event, then define corresponding actions for each severity level, as shown in FIGS. 12 and 13. FIG. 12 shows three sample events that each has a specific severity level. Thus, Event A has a severity level of Low; Event B has a severity level of Medium; and Event C has a severity level of High. Each severity level can then have corresponding actions defined, as shown in the table in FIG. 13. For events that have a severity level of “low”, the medical professional is notified and all related data is logged. For events that have a severity level of “medium”, the medical professional is notified, an administrator is notified, and all related data is logged. For events that have a severity level of “high”, the medical professional is notified, an administrator is notified, and all related data is logged.

Method 1400 in FIG. 14 is a method that is preferably performed by the decision support engine 123 in FIG. 1 and/or the medical decision support engine 280 in FIG. 2. System conditions and professional conditions are monitored (step 1410). When there is no event (step 1420=NO), method 1400 loops back to step 1410 and continues until an event occurs (step 1420=YES). Any applicable severity level and actions that correspond to the event are determined (step 1430). The actions corresponding to the event are then performed (step 1440). Method 1400 then loops back to step 1410 and continues.

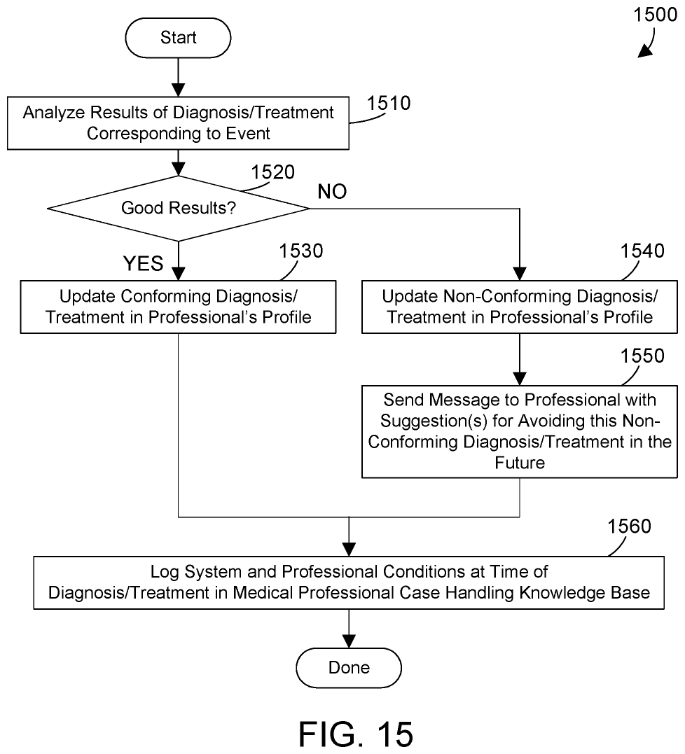

The results of a diagnosis or treatment that caused an event can be fed back to the system for future use. Referring to FIG. 15, a method 1500 is preferably performed by the decision support engine 123 in FIG. 1 and/or the medical decision support engine 280 in FIG. 2. The results of a diagnosis or treatment that caused an event are analyzed (step 1510). When the results were good (step 1520=YES), the diagnosis/treatment is added to the professional's profile (step 1530), such as in the conforming diagnoses/treatments 650 in FIG. 6. The system and professional conditions at the time of the diagnosis or treatment are logged in the medical professional case handling knowledge base (step 1560). When the results of the event were not good (step 1520=NO), the diagnosis/treatment is added to the professional's profile (step 1540), such as in the non-conforming diagnoses/treatments 660 in FIG. 6. A message could be sent to the medical professional with one or more suggestions to avoid this non-conforming diagnosis or treatment in the future (step 1550). The system and professional conditions at the time of the diagnosis or treatment are logged in the medical professional case handling knowledge base (step 1560). Method 1500 is then done.

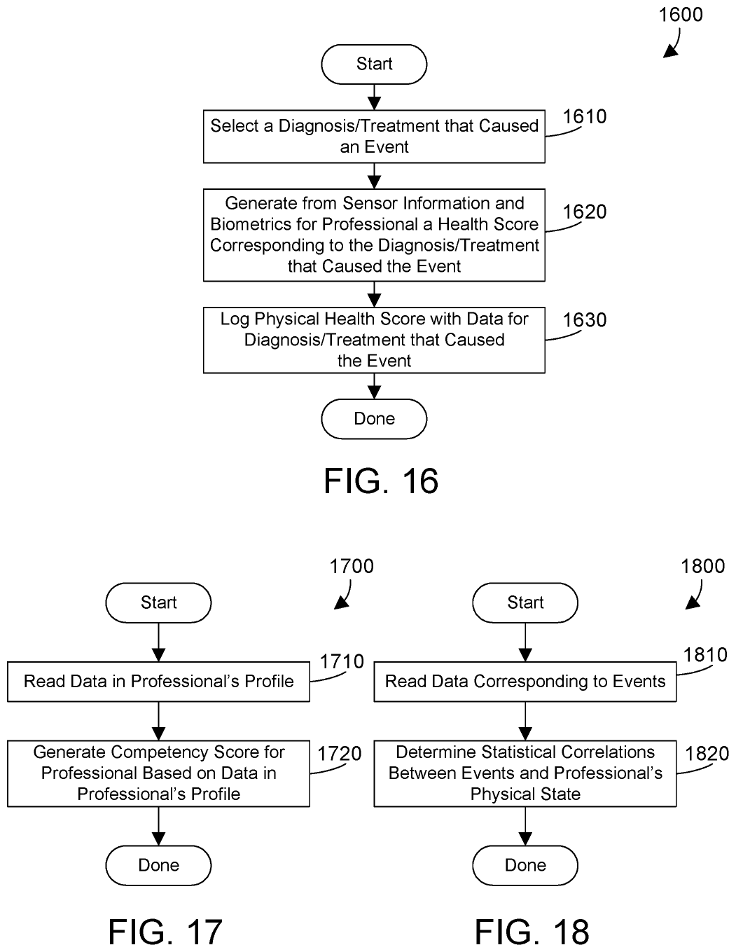

The ability to correlate both biometric and sensor data with events makes it possible to “score” the medical professional according to their current health state, providing a numerical indicator of the physical condition of the medical professional at the time the diagnosis or treatment was made. Referring to FIG. 16, a method 1600 is preferably performed by the decision support engine 123 in FIG. 1 and/or the medical decision support engine 280 in FIG. 2. A diagnosis or treatment that caused an event is selected (step 1610). From the logged sensor information and biometrics for the medical professional, a health score is generated which corresponds to the diagnosis or treatment that caused the event (step 1620). The physical health score of the medical professional is then logged as part of the data that corresponds to the diagnosis/treatment that caused the event (step 1630). Method 1600 is then done.

In addition to generating a health score for a medical professional for each event, an overall competency score can be generated based on data in the medical professional's profile relating to conforming and non-conforming diagnoses and treatments, such as 650 and 660 in FIG. 6. Referring to FIG. 17, a method 1700 is preferably performed by the decision support engine 123 in FIG. 1 and/or the medical decision support engine 280 in FIG. 2. Data in the medical professional's profile is read (step 1710). A competency score for the medical professional is then generated based on the data in the professional's profile (step 1720). Method 1700 is then done.

Because the biometric and sensor data is logged for each event, this logged data can be analyzed to determine any correlations that might exist. Referring to FIG. 18, a method 1800 is preferably performed by the decision support engine 123 in FIG. 1 and/or the medical decision support engine 280 in FIG. 2. The data corresponding to logged events is read (step 1810). Statistical correlations between events and the medical professional's physical state are determined (step 1820). Method 1800 is then done. By determining statistical correlations using method 1800, a medical organization such as a hospital could determine that fatigue tends to lead to events, and can therefore revise its policies regarding work and rest hours to assure their medical professionals are better rested.

The disclosure and claims herein support an apparatus comprising: at least one processor; a memory coupled to the at least one processor; and a decision support engine residing in the memory and executed by the at least one processor, the decision support engine comprising: a plurality of events that each comprises: a specification of at least one variance of a decision; and at least one corresponding action that includes providing a notification of the event to a person; a plurality of professional profiles that each corresponds to a plurality of professionals, wherein each of the plurality of professional profiles comprises decisions by the corresponding professional; a safety monitor tool that receives biometric data from biometric sensors worn by the plurality of professionals; wherein the decision support engine detects when one of the plurality of events occurs, and in response, provides the notification corresponding to the event to the person and logs the biometric data corresponding to the one professional that caused the one event to occur.

The disclosure and claims herein further support an apparatus comprising: at least one processor; a memory coupled to the at least one processor; and a medical treatment analysis engine residing in the memory and executed by the at least one processor, the medical treatment analysis engine comprising: a plurality of severity levels; a plurality of events that each comprises: a specification of at least one variance of a diagnosis or treatment; one of the plurality of severity levels; and at least one corresponding action that includes providing a notification of the event to a person; a plurality of professional profiles that each corresponds to a plurality of medical professionals, wherein each of the plurality of professional profiles comprises diagnoses and treatments by the corresponding medical professional, recent hours worked, recent sleep, recent events, and recent medications; a health monitor tool that receives biometric data from biometric sensors worn by the plurality of medical professionals, wherein the biometric data comprises heart rate, body temperature and a sleep log for the one medical professional that caused the one event to occur; a plurality of input sensors comprising video cameras, audio input devices and data input monitors that monitor interaction of the plurality of medical professionals with a plurality of patients; wherein the medical treatment analysis engine receives sensor data from the plurality of input sensors, wherein the sensor data comprises video streams, audio streams and typing speed; wherein the medical treatment analysis engine queries a medical cases knowledge base that correlates diagnoses with corresponding treatments and further comprising a work and rest regulations knowledge base that specifies regulations regarding amount of work and rest for medical professionals; wherein the medical treatment analysis engine queries a medical professional case handling knowledge base that includes a plurality of diagnoses and treatments, and for each diagnosis, corresponding biometric data for a medical professional that made the diagnosis, and for each treatment, corresponding biometric data for a medical professional that gave the treatment; wherein the medical treatment analysis engine detects when one of the plurality of events occurs, and in response, provides the notification corresponding to the event to the person and logs the biometric data corresponding to the one medical professional that caused the one event to occur, wherein the notification of the event to the person comprises at least one of: notifying the medical professional; notifying a patient corresponding to the event; and notifying an administrator.

The disclosure and claims herein additionally support a computer-implemented method executed by at least one processor for detecting variance in decisions by professionals, the method comprising: defining a plurality of events that each comprises: a specification of at least one variance of a decision; and at least one corresponding action that includes providing a notification of the event to a person; providing and maintaining a plurality of professional profiles that each corresponds to a plurality of professionals, wherein each of the plurality of professional profiles comprises decisions by the corresponding professional; receiving biometric data from biometric sensors worn by the plurality of professionals; detecting when one of the plurality of events occurs, and in response: providing the notification corresponding to the event to the person; and logging the biometric data corresponding to the one professional that caused the one event to occur.

A decision support engine allows defining multiple events and corresponding actions for those events. An event could be, for example, a decision that is at variance with what the professional usually suggests, or at variance with standard practice of other professionals. Suitable action can be taken when an event occurs. Examples of suitable actions include, for example, to notify the professional, to notify the customer or patient, to notify an administrator, and to log all data related to the decision that caused the event. The decision support engine receives biometric input from one or more biometric sensors worn by the professional, and can thus log the physical state of the professional at the time the event occurs. Both conforming and non-conforming decisions can be logged to a profile maintained for the professional, along with the corresponding biometric data. For each event, the professional's biometric data is logged and can be analyzed to determine statistical correlations between the events corresponding to the professional and the professional's physical state.

One skilled in the art will appreciate that many variations are possible within the scope of the claims. Thus, while the disclosure is particularly shown and described above, it will be understood by those skilled in the art that these and other changes in form and details may be made therein without departing from the spirit and scope of the claims.

1. An apparatus comprising:
at least one processor;
a memory coupled to the at least one processor; and
a decision support engine residing in the memory and executed by the at least one processor, the decision support engine comprising:
a plurality of events that each comprises:
a specification of at least one variance of a decision; and
at least one corresponding action that includes providing a notification of the event to a person;
a plurality of professional profiles that each corresponds to a plurality of professionals, wherein each of the plurality of professional profiles comprises decisions by the corresponding professional;
a safety monitor tool that receives biometric data from biometric sensors worn by the plurality of professionals;
wherein the decision support engine detects when one of the plurality of events occurs, and in response, provides the notification corresponding to the event to the person and logs the biometric data corresponding to the one professional that caused the one event to occur.

2. The apparatus of claim 1 wherein the biometric data comprises heart rate, body temperature and a sleep log for the one professional that caused the one event to occur.

3. The apparatus of claim 1 wherein the decision support engine receives sensor data from a plurality of input sensors that monitor interaction of the plurality of professionals, wherein the sensor data comprises video streams, audio streams and typing speed.

4. The apparatus of claim 3 wherein the plurality of input sensors comprises video cameras, audio input devices and data input monitors.

5. The apparatus of claim 1 wherein the decision support engine queries at least one knowledge base comprising a medical cases knowledge base that correlates diagnoses with corresponding treatments and further comprising a work and rest regulations knowledge base that specifies regulations regarding amount of work and rest for medical professionals.

6. The apparatus of claim 5 wherein the at least one knowledge base comprises a medical professional case handling knowledge base that includes a plurality of diagnoses and treatments, and for each diagnosis, corresponding biometric data for a medical professional that made the diagnosis, and for each treatment, corresponding biometric data for a medical professional that gave the treatment.

7. The apparatus of claim 1 wherein each of the plurality of professional profiles comprises recent hours worked, recent sleep, recent events, and recent medications.

8. The apparatus of claim 1 further comprising a plurality of severity levels, wherein each of the plurality of events specifies a corresponding one of the plurality of severity levels.

9. The apparatus of claim 1 wherein notification of the event to the person comprises at least one of: notifying the professional; notifying a patient corresponding to the event;
and notifying an administrator.

10. An apparatus comprising:
at least one processor;
a memory coupled to the at least one processor; and
a medical treatment analysis engine residing in the memory and executed by the at least one processor, the medical treatment analysis engine comprising:
a plurality of severity levels;
a plurality of events that each comprises:
a specification of at least one variance of a diagnosis or treatment;
one of the plurality of severity levels; and
at least one corresponding action that includes providing a notification of the event to a person;
a plurality of professional profiles that each corresponds to a plurality of medical professionals, wherein each of the plurality of professional profiles comprises diagnoses and treatments by the corresponding medical professional, recent hours worked, recent sleep, recent events, and recent medications;
a health monitor tool that receives biometric data from biometric sensors worn by the plurality of medical professionals, wherein the biometric data comprises heart rate, body temperature and a sleep log for the one medical professional that caused the one event to occur;
a plurality of input sensors comprising video cameras, audio input devices and data input monitors that monitor interaction of the plurality of medical professionals with a plurality of patients;
wherein the medical treatment analysis engine receives sensor data from the plurality of input sensors, wherein the sensor data comprises video streams, audio streams and typing speed;
wherein the medical treatment analysis engine queries a medical cases knowledge base that correlates diagnoses with corresponding treatments and further comprising a work and rest regulations knowledge base that specifies regulations regarding amount of work and rest for medical professionals;
wherein the medical treatment analysis engine queries a medical professional case handling knowledge base that includes a plurality of diagnoses and treatments, and for each diagnosis, corresponding biometric data for a medical professional that made the diagnosis, and for each treatment, corresponding biometric data for a medical professional that gave the treatment;
wherein the medical treatment analysis engine detects when one of the plurality of events occurs, and in response, provides the notification corresponding to the event to the person and logs the biometric data corresponding to the one medical professional that caused the one event to occur, wherein the notification of the event to the person comprises at least one of: notifying the medical professional; notifying a patient corresponding to the event; and notifying an administrator.

11. A computer-implemented method executed by at least one processor for detecting variance in decisions by professionals, the method comprising:
defining a plurality of events that each comprises:
a specification of at least one variance of a decision; and
at least one corresponding action that includes providing a notification of the event to a person;
providing and maintaining a plurality of professional profiles that each corresponds to a plurality of professionals, wherein each of the plurality of professional profiles comprises decisions by the corresponding professional;
receiving biometric data from biometric sensors worn by the plurality of professionals;
detecting when one of the plurality of events occurs, and in response:
providing the notification corresponding to the event to the person; and
logging the biometric data corresponding to the one professional that caused the one event to occur.

12. The method of claim 11 wherein the biometric data comprises heart rate, body temperature and a sleep log for the one professional that caused the one event to occur.

13. The method of claim 11 further comprising:
receiving sensor data from a plurality of input sensors that monitor interaction of the plurality of professionals, wherein the sensor data comprises video streams, audio streams and typing speed.

14. The method of claim 13 wherein the plurality of input sensors comprises video cameras, audio input devices and data input monitors.

15. The method of claim 11 further comprising:
querying at least one knowledge base comprising a medical cases knowledge base that correlates diagnoses with corresponding treatments and further comprising a work and rest regulations knowledge base that specifies regulations regarding amount of work and rest for medical professionals.

16. The method of claim 15 wherein the at least one knowledge base comprises a medical professional case handling knowledge base that includes a plurality of diagnoses and treatments, and for each diagnosis, corresponding biometric data for a medical professional that made the diagnosis, and for each treatment, corresponding biometric data for a medical professional that gave the treatment.

17. The method of claim 11 wherein each of the plurality of professional profiles comprises recent hours worked, recent sleep, recent events, and recent medications.

18. The method of claim 11 further comprising:
defining a plurality of severity levels, wherein each of the plurality of events specifies a corresponding one of the plurality of severity levels.

19. The method of claim 11 wherein providing the notification of the event to the person comprises at least one of: notifying the professional; notifying a patient corresponding to the event; and notifying an administrator.
